(19) United States
(12) Patent Application Publication
(10) Pub. No.: US 2020/0000252 A1
(43) Pub. Date: Jan. 2, 2020

(54) WARMING BLANKET

(71) Applicant: KAISER TECHNOLOGY CO., LTD., New Taipei City (TW)

(72) Inventor: Mu-Jen WANG, New Taipei City (TW)

(21) Appl. No.: 16/138,594

(22) Filed: Sep. 21, 2018

(30) Foreign Application Priority Data
Jun. 29, 2018 (CN) 201810696935.6

(51) Int. Cl. A47G 9/02; A61F 7/00; A61F 7/08

(52) U.S. Cl. CPC A47G 9/0215; A61F 2007/006; A61F 7/08; A61F 7/0097

(57) ABSTRACT

A warming blanket includes a warm-air generator, a blanket unit, an air-providing tube and a returning tube. The warm-air generator includes a blower that has an air inlet for drawing air, and a heating member for heating air. The blanket unit has first and second air channels. The warm air which is heated by the heating member is transmitted into the first air channel through the air-providing tube, flows through the first and second air channels, and is transmitted back to the air inlet of the blower.

### CROSS-REFERENCE TO RELATED APPLICATION

This application claims priority of Chinese Patent Application No. 201810696935.6, filed on Jun. 29, 2018.

### FIELD

The disclosure relates to a warming blanket, more particularly to a warming blanket used to maintain the core temperature of a patient.

### BACKGROUND

Warming blankets are used as a source of heat for patients undergoing surgical procedures. It safely and effectively warms up the patient so as to minimize the risks of the surgery by preventing excessive loss of body heat from the patient during surgery.

A conventional warming blanket generally expels heated air from a bottom thereof. This heated air may circulate around the operating table, mixing with ambient air that is more likely to have bacteria and thus increasing risks of infection.

Furthermore, since the conventional warming blanket expels all of the heated air, new air needs to be reheated constantly and energy is not used efficiently.

Another device, for example, a cold and warm circulatory blanket disclosed in Taiwanese Utility Model Patent No. M542128 and including a blanket, a control system and an external device, would also experience the aforementioned issues when applied in medical warming uses.

### SUMMARY

Therefore, in a first aspect, this disclosure provides a warming blanket that can alleviate at least one of the drawbacks of the prior art.

Accordingly, the warming blanket includes a warm-air generator, a blanket unit, an air-providing tube and a returning tube.

The warm-air generator includes a housing, a blower that is disposed in the housing, and a heating member. The blower has an air inlet for drawing air therethrough into the housing, and an outlet tube. The heating member is disposed in the housing for heating the air advanced from the outlet tube.

The blanket unit has a first tube-connecting member, a first air channel that is connected to the first tube-connecting member, a second air channel that is serpentine and that is connected to the first air channel, and a second tube-connecting member that is connected to the second air channel.

The air-providing tube is connected between the outlet tube of the blower and the first tube-connecting member of the blanket unit.

The returning tube is connected between the second tube-connecting member of the blanket unit and the air inlet of the blower.

The warm air which is heated by the heating member is transmitted into the first air channel through the air-providing tube, flows through the first and second air channels, and is transmitted back to the air inlet of the blower.

In a second aspect, this disclosure also provides a warm air generator adapted to be connected to an air-providing tube of a warming blanket.

Accordingly, the warm-air generator includes a housing, a blower, a heating member and a guide seat.

The blower is disposed in the housing, and has an air inlet for drawing air therethrough into the housing, and an outlet tube.

The heating member is disposed in the housing and is for heating the air advanced from the outlet tube.

The guide seat includes a shell body, an inner tube, and a connecting tube. The shell body is connected to the outlet tube of the blower, and defines a receiving space therein. The inner tube that is funnel-shaped, and has a connecting open end that is connected to the shell body and that blocks a space between the inner tube and the shell body, and a distal open end that is smaller than the connecting open end and that is inserted into the receiving space. The inner tube is adapted to be in fluid communication with the outlet tube of the blower and the air-providing tube. The connecting tube is connected to the shell body, and is in fluid communication with the receiving space and the air inlet of the blower.

### BRIEF DESCRIPTION OF THE DRAWINGS

Other features and advantages of the disclosure will become apparent in the following detailed description of the embodiments with reference to the accompanying drawings, of which:

FIG. 1 is a perspective view of a first embodiment of a warming blanket according to the disclosure;

FIG. 2 is an exploded perspective view of the first embodiment;

FIG. 3 is a fragmentary sectional view of the first embodiment;

FIG. 4 is another fragmentary sectional view of the first embodiment;

FIG. 5 is a sectional view taken along line V-V in FIG. 3;

FIG. 6 is a sectional view taken along line VI-VI in FIG. 3;

FIG. 7 is a partly cutaway perspective view of a guide seat of the first embodiment; and

FIG. 8 is a perspective view of a second embodiment of the warming blanket according to the disclosure.

### DETAILED DESCRIPTION

Before the present invention is described in greater detail, it should be noted that where considered appropriate, reference numerals or terminal portions of reference numerals have been repeated among the figures to indicate corresponding or analogous elements, which may optionally have similar characteristics.

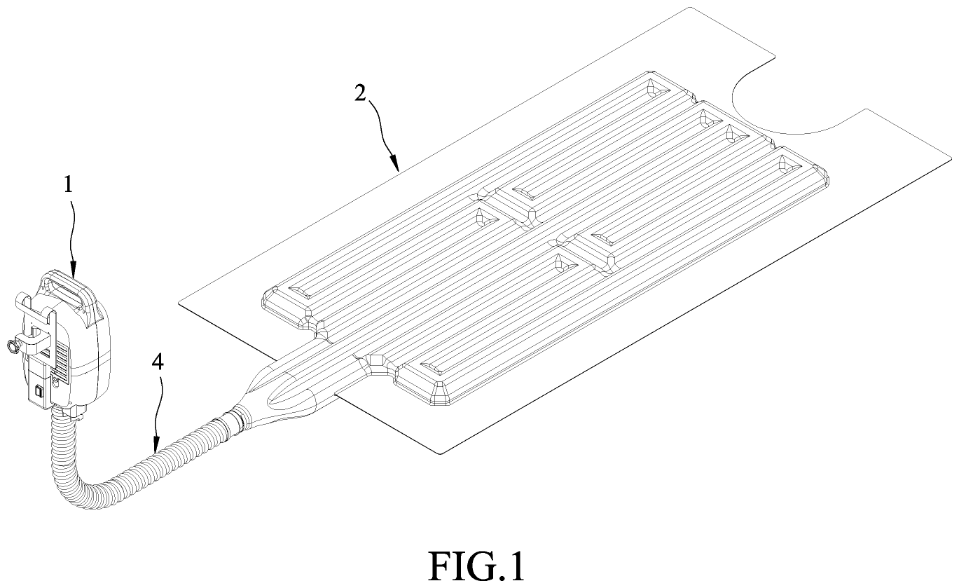

Referring to FIG. 1, a first embodiment of a warming blanket according to the disclosure includes a warm-air generator 1, a blanket unit 2, an air-providing tube 3 connected between the warm-air generator 1 and the blanket unit 2, and a returning tube 4 connected between the warm-air generator 1 and the blanket unit 2.

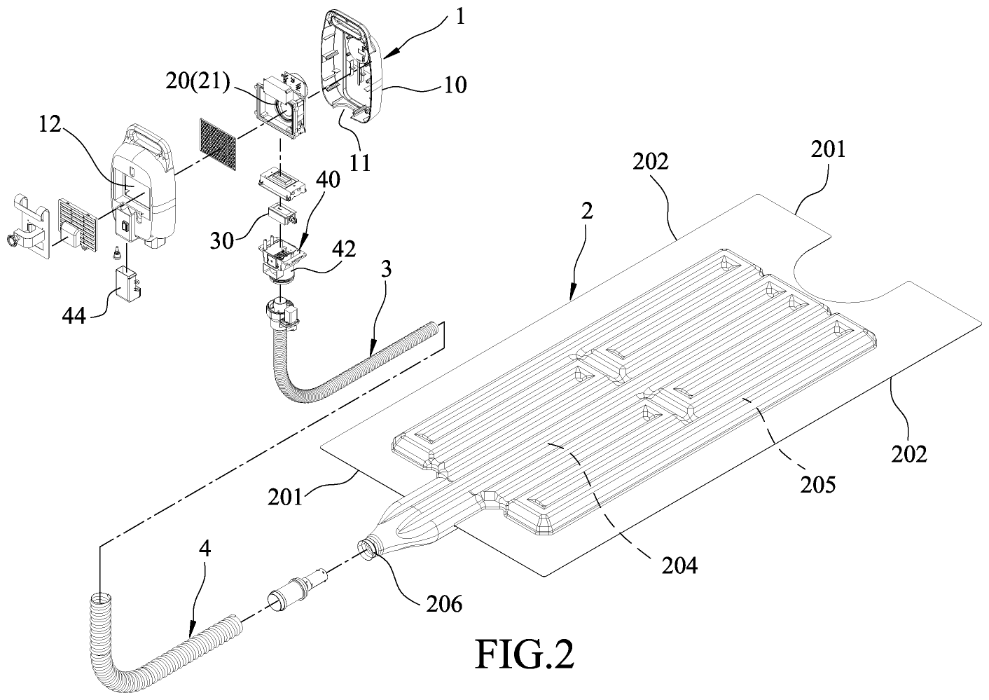

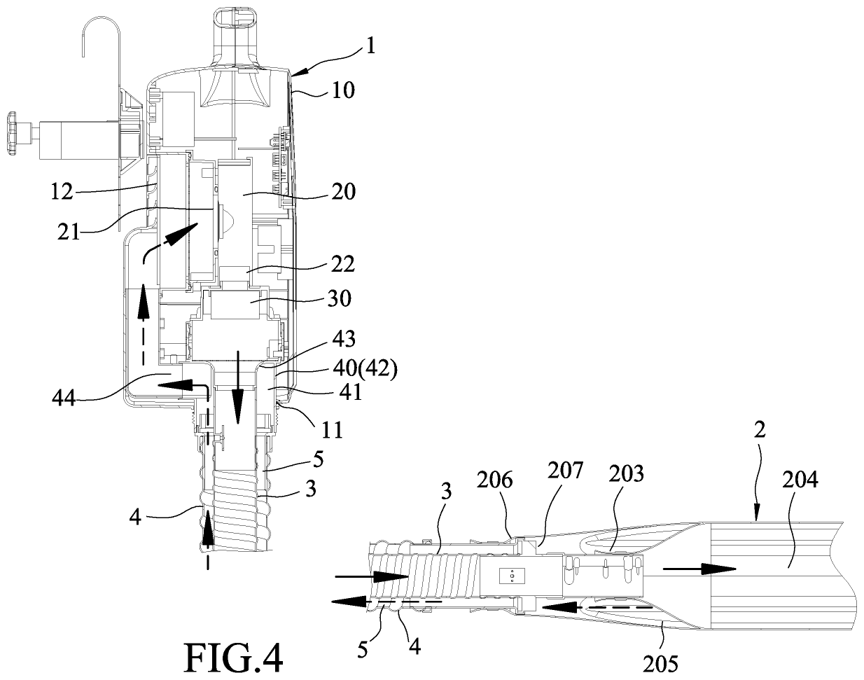

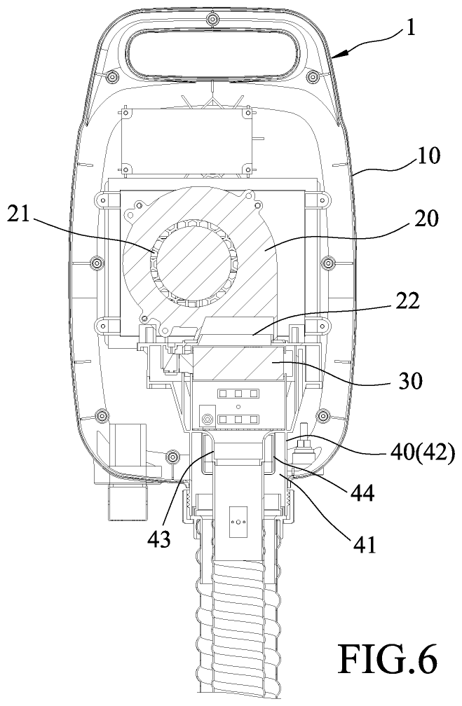

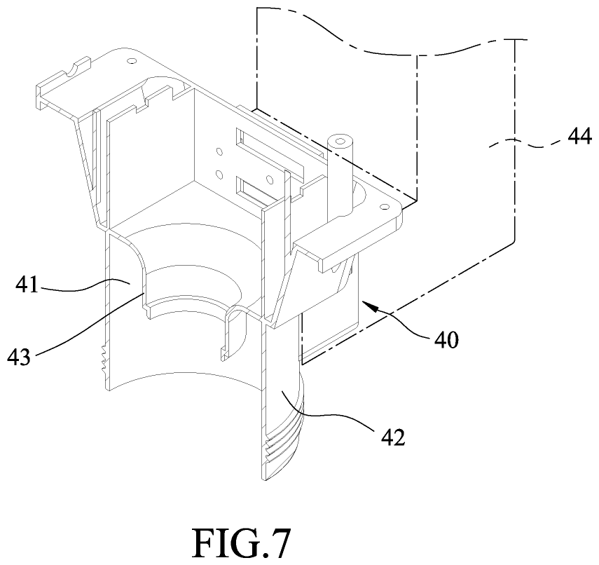

Referring to FIGS. 2, 3, 4, and 6, the warm-air generator 1 includes a housing 10, a blower 20 that is disposed in the housing 10, a heating member 30 that is disposed in the housing 10 and on one side of the blower 20, and a guide seat 40 that is connected to the blower 20. The housing 10 is formed with a through hole 11 in a bottom thereof, and has a external inlet 12 in a back thereof. The blower 20 has an air inlet 21 that is disposed for drawing air therethrough into the housing 10 and that is proximal to the external inlet 12, and an outlet tube 22 that is disposed at one side of the air inlet 21. The heating member 30 is connected to the outlet tube 22 for heating the air advanced from the outlet tube 22. Referring further to FIGS. 4 and 7, the guide seat 40 includes a shell body 42 that is connected to the outlet tube 20 and that defines a receiving space 41, an inner tube 43 that extends within the receiving space 41 and that is connected to the shell body 42, and a connecting tube 44 that is connected to the back of the shell body 42. The inner tube 43 is funnel-shaped, and has a connecting open end connected to the shell body 42 and blocking a space between the inner tube 43 and the shell body 42, and a distal open end smaller than the connecting open end and inserted into the receiving space 41. The inner tube 43 is in fluid communication with the outlet tube 22. The heating member 30 is disposed between the inner tube 43 and the outlet tube 22, and the connecting tube 44 is connected between and in fluid communication with the receiving space 41 and the air inlet 21.

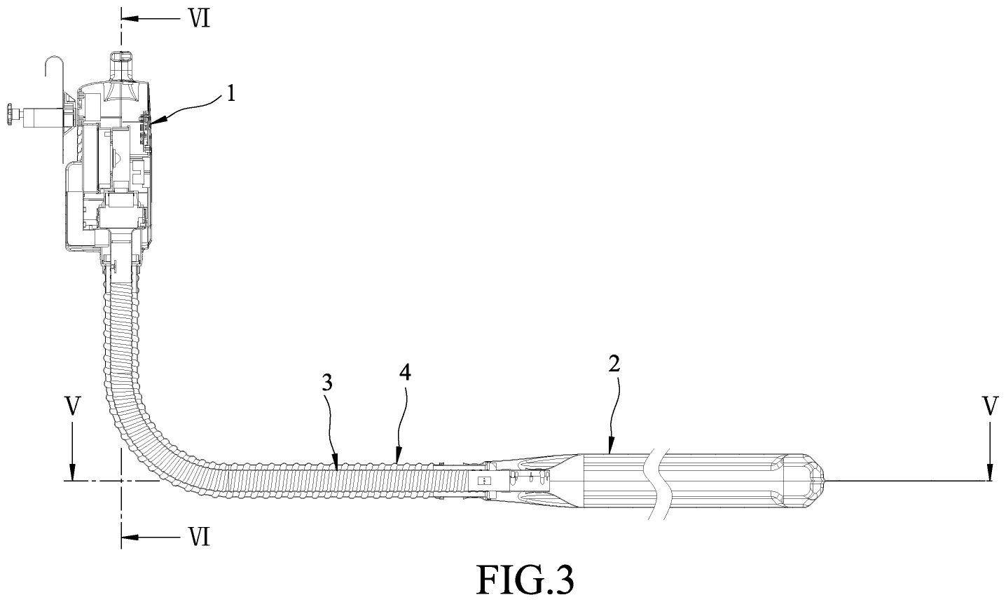

Referring to FIGS. 3 to 5, in this embodiment, the blanket unit 2 has two opposite short edges 201, two opposite long edges 202 that interconnect the short edges 201, a first tube-connecting member 203 that is disposed on one of the short edges 201, a first air channel 204 that is disposed internally and connected to the first tube-connecting member 203, two second air channels 205 that are connected to the first air channel 204 and that are serpentine, and a second tube-connecting member 206 that is disposed on the one of the short edges 201 and that is connected to both of the second air channels 205. In this embodiment, the first tube-connecting member 203 is disposed in and extends through the second tube-connecting member 206, and cooperates with the second tube-connecting member 206 to define an annular air passage 207 that is in fluid communication with the second air channel 205.

Referring to FIGS. 3 and 4, the air-providing tube 3 is connected between the inner tube 43 and the first tube-connecting member 203 (and thus connected between the outlet tube 22 of the blower 20 and the first tube-connecting member 203 of the blanket unit 2) and transmits the warm air which is heated by the heating member 30 into the first air channel 204 via the first tube-connecting member 203.

Referring to FIGS. 3 and 4, the returning tube 4 is connected between the shell body 42 and the second tube-connecting member 206 (and thus connected between the air inlet 21 and the second tube-connecting member 206), and transmits the air from the second air channels 205 back to the warm-air generator 1 by returning the air from the blanket unit 2 through the receiving space 41 and the connecting tube 44 back into the air inlet 21, thus circulating and reusing the warm air.

In this embodiment, the air-providing tube 3 is disposed within the returning tube 4, and the air-providing tube 3 and the returning tube 4 cooperatively define a returning channel 5 which is in fluid communication with the receiving space 41 and the air passage 207.

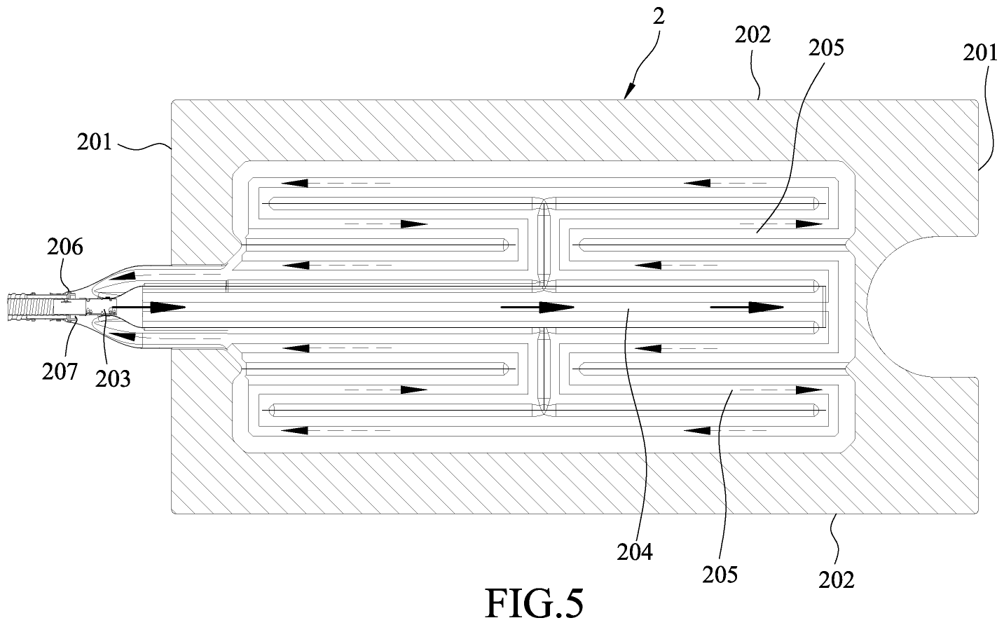

As shown in FIGS. 1, 4, 5, and 6, when the warm-air generator 1 is switched on and the blower 20 is activated, air is drawn from the air inlet 21 and transmitted through the outlet tube 22. The heating member 30 heats the air advanced from the outlet tube 22, after which the warm air is transmitted through the inner tube 43 into the air-providing tube 3, and then transmitted into the first air channel 204 through the first tube-connecting member 203, along the route as indicated by solid arrows in FIGS. 4 and 5. The air flows sinuously through the second air channels 205 and the air passage 207, and returns to the air inlet 21 through the returning channel 5 and the connection tube 44, along the route indicated by the dashed arrows. The flow of the warm air through the first air channel 204 and second air channels 205 provides optimized warming to a patient in surgery, maintaining the core temperature of the patient.

Overall, the warming blanket according to the disclosure provides the following benefits:

1. As the warm air is in closed circulation within the first air channel 204 and the second air channel 205, the drawbacks associated with the abovementioned prior art, i.e., the interaction between the warm air and the ambient air, can be avoided, thereby relatively lowering the risk of infections.

2. As the warm air is in closed circulation within the first air channel 204 and the second air channel 205, the warm air is not expelled, thereby improving the comfort of the medical staff.

3. The warm air is reused, thereby improving energy efficiency.

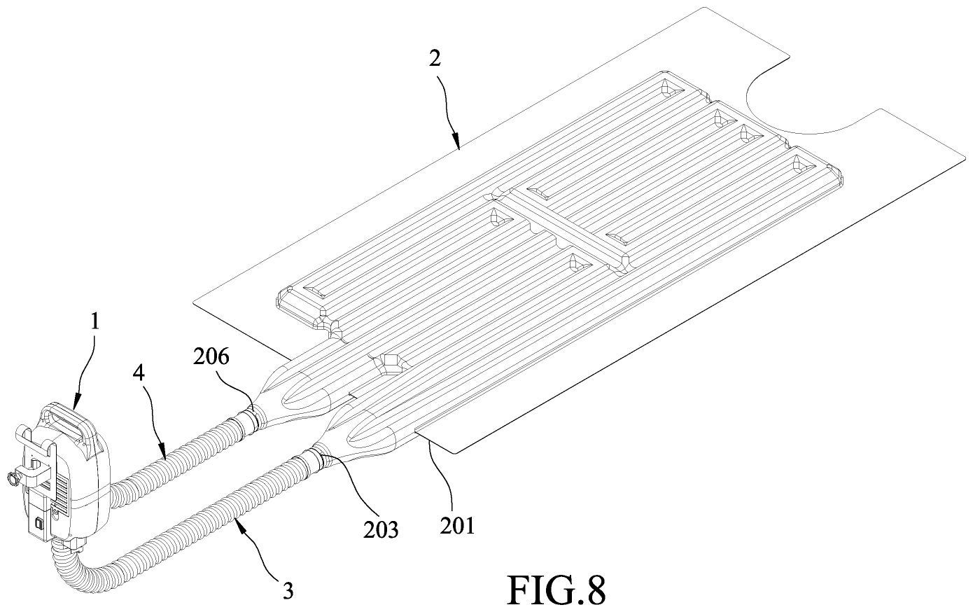

Referring to FIG. 8, a second embodiment of the warming blanket according to this disclosure has a structure similar to that of the first embodiment. The notable differences between the first embodiment and the second embodiment are that the first tube-connecting member 203 and the second tube-connecting member 206 are spaced apart from each other and are disposed side by side on one of the short edges 201, and that the air-proving tube 3 and the returning tube 4 are spaced apart from each other and respectively connected to the first tube-connecting member 203 and the second tube-connecting member 206. The second embodiment has the same benefits as the first embodiment.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A warming blanket comprising:
a warm-air generator including
a housing,
a blower that is disposed in said housing, and that has an air inlet for drawing air therethrough into said housing, and an outlet tube, and
a heating member that is disposed in said housing for heating the air advanced from said outlet tube;
a blanket unit having
a first tube-connecting member,
a first air channel that is connected to said first tube-connecting member,
a second air channel that is serpentine and that is connected to said first air channel, and
a second tube-connecting member that is connected to said second air channel;
an air-providing tube connected between said outlet tube of said blower and said first tube-connecting member of said blanket unit; and
a returning tube connected between said second tube-connecting member of said blanket unit and said air inlet of said blower;
wherein the warm air which is heated by said heating member is transmitted into said first air channel through said air-providing tube, flows through said first and second air channels, and is transmitted back to said air inlet of said blower.

2. The warming blanket as claimed in claim 1, wherein:
said blanket unit further has two opposite short edges, and two opposite long edges that interconnect said short edges;
said first and second tube-connecting members are disposed on one of said short edges;
said first tube-connecting member is disposed in and extends through said second tube-connecting member, and cooperates with said second tube-connecting member to define an annular air passage that is in fluid communication with said second air channel; and
said air-providing tube and said returning tube cooperatively define a returning channel therebetween that is in fluid communication with said air passage.

3. The warming blanket as claimed in claim 2, wherein said warm-air generator further includes a guide seat including:
a shell body that is connected to said outlet tube of said blower, and that defines a receiving space therein;
an inner tube that is funnel-shaped, that has a connecting open end connected to said shell body and blocking a space between said inner tube and said shell body, and a distal open end smaller than said connecting open end and inserted into said receiving space, said inner tube being in fluid communication with said outlet tube of said blower and said air-providing tube; and
a connecting tube that is connected to said shell body, and that is in fluid communication with said receiving space and said air inlet of said blower.

4. The warming blanket as claimed in claim 1, wherein:
said blanket unit further has two opposite short edges, and two opposite long edges that interconnect said short edges;
said first and second tube-connecting members are disposed on one of said short edges and are spaced apart from each other; and
said air-providing tube and said returning tube are spaced apart from each other.

5. A warm-air generator adapted to be connected to an air-providing tube of a warming blanket, said warm-air generator comprising:
a housing;
a blower that is disposed in said housing, and that has an air inlet for drawing air therethrough into said housing, and an outlet tube;
a heating member that is disposed in said housing for heating the air advanced from said outlet tube; and
a guide seat that includes
a shell body connected to said outlet tube of said blower, and defining a receiving space therein,
an inner tube being funnel-shaped, having a connecting open end that is connected to said shell body and that blocks a space between said inner tube and said shell body, and a distal open end that is smaller than said connecting open end and that is inserted into said receiving space, said inner tube being adapted to be in fluid communication with said outlet tube of said blower and the air-providing tube, and
a connecting tube connected to said shell body, and being in fluid communication with said receiving space and said air inlet of said blower.
